(19) United States
(12) Patent Application Publication
(10) Pub. No.: US 2024/0269106 A1
(43) Pub. Date: Aug. 15, 2024

(54) SMALL MOLECULE MODULATOR TARGETING A RARE HISTONE MODIFICATION REGULATING ADIPOGENESIS AND PHARMACEUTICAL FORMULATION THEREOF

(71) Applicants: COUNCIL OF SCIENTIFIC AND INDUSTRIAL RESEARCH AN INDIAN REGISTERED BODY INCORPORATED UNDER THE REGN, New Delhi, Delhi (IN); JAWAHARLAL NEHRU CENTRE FOR ADVANCED SCIENTIFIC RESEARCH, Jakkur, Bangalore (IN)

(72) Inventors: Tapas Kumar KUNDU, Jakkur, Karnataka, Bangalore (IN); Aditya BHATTACHARYA, Jakkur, Karnataka, Bangalore (IN); Sourav CHATTERJEE, Jakkur, Karnataka, Bangalore (IN); Venkata Sashidhara KONENI, Uttar Pradesh, Lucknow (IN); Suriya Pratap SINGH, Uttar Pradesh, Lucknow (IN); Prabhat Ranjan MISHRA, Uttar Pradesh, Lucknow (IN); Aamir NAZIR, Uttar Pradesh, Lucknow (IN); Rajdeep GUHA, Uttar Pradesh, Lucknow (IN)

(21) Appl. No.: 18/565,911

(22) PCT Filed: Jun. 2, 2022
(86) PCT No.: PCT/IN2022/050515

§ 371 Date: Nov. 30, 2023

(30) Foreign Application Priority Data
Jun. 2, 2021 (IN) 202111024677

(51) Int. Cl. A61K 31/352; A61K 9/00; A61K 36/38; A61K 47/10; A61K 47/14

(52) U.S. Cl. CPC A61K 31/352; A61K 9/0053; A61K 36/38; A61K 47/10; A61K 47/14; A61K 2236/30

(57) ABSTRACT

The present invention relates to substituted benzophenones of structural Formula I employed as small molecule inhibitor for controlling obesity. The present invention further relates to screening of small molecule inhibitors against p300 which selectively targets histone butyrylation thereby specifically inhibiting adipogenesis further aiding to prevention of weight gain. The present invention reveals that histone butyrylation increases during adipogenesis. Hence, inhibition of histone butyrylation by a small molecule inhibitor would be a promising therapeutic strategy to control obesity. Significantly, besides inhibiting adipogenesis in cellular model, it also prevents weight gain in high fat diet based mouse model system, indicating the possible use of the scaffold for obesity control.

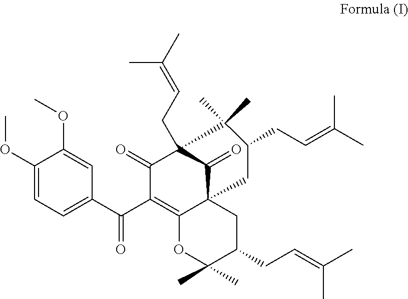

### FIELD OF THE INVENTION

The present invention relates to substituted benzophenone a small molecule inhibitor for controlling obesity.

The present invention particularly relates to substituted benzophenone of structural Formula I, a small molecule inhibitor for controlling obesity.

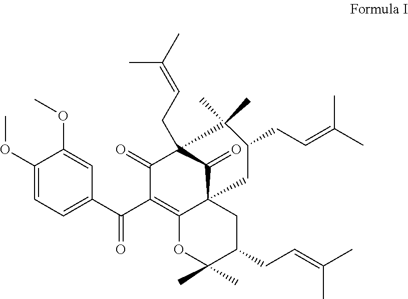

The present invention further relates to a small molecule inhibitor that selectively targets p300 catalysed histone butyrylation without affecting acetylation and thereby specifically inhibiting adipogenesis.

### BACKGROUND OF THE INVENTION

Since the last decade, owing to major advances in high sensitivity mass spectrometry, more and more new post-translational modifications are being discovered (Chen, Y et al, Lysine propionylation and butyrylation are novel post-translational modifications in histones. Mol Cell Proteomics 6, 812-819 (2007)). Of particular interest is a group of acylation modifications which structurally closely resemble acetylation, but differ in hydrocarbon chain length, hydrophobicity and charge. Emerging evidences in recent reports indicate that these modifications occur dynamically on histones and could either have distinct functions from acetylation, or, have a similar but more profound impact on chromatin de-compaction, thereby amplifying the transcriptional output. Till now, p300 is the major acyltransferase that has been found to promiscuously acylate histones using diverse coenzyme (CoA) derivatives. By virtue of the presence of a hydrophobic pocket to accommodate the variable aliphatic chain of the acyl CoA, p300 can catalyze these additional modifications albeit at a slower rate than acetylation (Kaczmarska et al, Structure of p300 in complex with acyl-CoA variants. Nat Chem Biol 13, 21-29 (2017)).

Till date, there have been very few reports on the physiological relevance of these rarer acylation modifications. Recent study has shown that histone butyrylation at H4K5 and H4K8 positions are more stable marks compared to histone acetylation and are removed later compared to acetylation in the late stages of spermatogenesis (Goudarzi et al., Dynamic Competing Histone H4 K5K8 Acetylation and Butyrylation Are Hallmarks of Highly Active Gene Promoters. Mol Cell 62, 169-180 (2016)). Because the acetylated histones are removed by BRDT (a testes specific Bromodomain containing protein) which exhibits reduced affinity for H4 peptides butyrylated at H4K5 position, this finding indicates that H4K5 butyrylation might mark those regions of the genomic loci that retain histones throughout the spermatogenesis process. Interestingly, under starvation conditions, when the liver starts synthesizing β-hydroxybutyrate to meet the rising energy demand, the increased levels of β-hydroxybutyrate leads to β-hydroxybutyrylation of histones, especially in the promoters of starvation inducible genes (Xie, Z., et al., Metabolic Regulation of Gene Expression by Histone Lysine β-Hydroxybutyrylation. Mol Cell 62, 194-206 (2016)). It has also been demonstrated that lysine crotonylation of histones increases in LPS (lipopolysaccharide) mediated inflammatory response and Af9 is a reader domain protein that recognizes the crotonylation mark to carry out its downstream effects (Sabari et al., Intracellular crotonyl-CoA stimulates transcription through p300-catalyzed histone crotonylation. Mol Cell 58, 203-215 (2015)). Very recently, lysine propionylation has been reported as an activation mark on histones that could be predictably useful in various physiological processes (Kebede et al., Histone propionylation is a mark of active chromatin, Nat Struct Mol Biol 24, 1048-1056 (2017)).

Butyryl CoA is a metabolic intermediate in fatty acid breakdown/synthesis processes. Therefore it has been hypothesized that the process of adipogenesis in which lipid metabolism is predominant, might be epigenetically regulated by lysine butyrylation of histones. In this invention it has been opted to further highlight the role of histone butyrylation in adipogenesis by adopting a chemical biology approach. This in turn, could establish a connection of this work with human healthcare. The present invention describes the connection of p300 catalyzed histone butyrylation with adipogenesis. This invention also presents a polyisoprenylated benzophenone compound of general Formula I having a structure as defined above. This compound x selectively inhibits p300 catalyzed histone butyrylation without affecting acetylation. Subsequent cellular and animal studies depicted that the compound x has the potential to inhibit adipocyte differentiation by selective inhibition of butyrylation, thereby controlling adipogenesis.

Previously, there have been a few findings with structurally similar molecules in other aspects of biology. Yuan et. al., 2018 in CN 109020945 A, discussed about certain isogarcinol derivatives useful for the treatment of cancer. Lin et. al., 2019 discussed the synthesis of mixture of guttiferone E and xanthochymol derivatives an inseparable polycyclic polyprenylated acylphloroglucinol (PPAP), showing moderate cytotoxic activities by inducing cell apoptosis and arresting the cell cycle phase, specifically projecting to target as an anti-leukemia agent. Mantelingu et al., Specific inhibition of p300-HAT alters global gene expression and represses HIV replication. Chem Biol 14, 645-657 (2007), described the synthesis and characterization of a set of p300-HAT-specific small molecule inhibitors from a natural nonspecific HAT inhibitor, garcinol, which is highly toxic to cells. However, these inhibitors were found to be nontoxic to T cells, they could inhibit histone acetylation in HIV infected cells, and consequently inhibit the multiplication of HIV. Rama Rao, A. V et. al., 1979 and 1980 discussed the spectral data of isoxanthochymol, from Garcinia xanthochymus. Further Sei, Janet et. al., 2020 in WO 2020077343 A1 described preparation of a growth medium for culture of hematopoietic stem cells (HSCs), consisting of a basal medium and a supplement with histone acetyltransferase (HAT) inhibitor, a histone deacetylase (HDAC) inhibitor and further discussed the methods of using the growth medium, as well as kits and formulations of the growth medium. Ansari, Aseem et. al. 2019 in WO 2019217757 A1 disclosed how similar compounds could be used for modulating the expression of c9orf72 (brain expressed, associated with NEDD4) and treating diseases and conditions in which c9orf72 plays an active role. Ovalle-Magallanes et. al. had described in a review article in 2017 ((Food and Chemical Toxicology (2017), 109(Part 1), 102-122) the potential anti-obesity property of structurally similar phytochemical-polyisoprenylated xanthones, a secondary metabolite found in mangosteen fruits. However, no details of exhaustive experimentation, which the present invention described as specifically targeting histone butyrylation in regulation of adipogenesis, have been disclosed. Ullrich, Evelyn et. al., 2016 in WO 2016020434 A1 disclosed a method for the in-vitro or ex-vivo expansion of immune cells for use in adoptive immunotherapy. The method comprises the use of a histone acetyl transferase (HAT) modulator as additive in the cell culture medium. Shen et. al. demonstrated in his publication in 2015 (Molecular Cancer Therapeutics (2015), 14(7), 1738-1749) that a similar molecule called cambogin, a polycyclic polyprenylated acylphoroglucinol (PPAP) from the Garcinia genus, inhibited breast cancer cell proliferation and induced cell apoptosis in vitro. Collins et. al. described in his publication in 2013 (BMC Cancer (2013), 13, 37.) that treatment of MCF7, SaOS2 and U20S cell lines with garcinol led to a decrease in H3K18 acetylation which is required for S phase progression and therefore led to an arrest in cell proliferation. In addition, garcinol was found to block CBP/p300-mediated acetylation of the C-terminal activation domain of p53, but resulted in enhanced acetylation of p53K120, and accumulation of p53 in the cytoplasmic compartment. Majeed, Muhammed in his patent US 20120178801 A1 20120712 disclosed anti-obesity property of two similar compounds isoxanthochymol and isogarcinol. Baliga et. al. in a review article (Food Research International (2011), 44(7), 1790-1799) described the medicinal uses of Garcinia indica Choisy in several contexts including its application in obesity treatment. DalPiaz et. al. disclosed in his publication in 2010 (ChemBioChem (2010), 11(6), 818-827) that the polyisoprenylated benzophenone derivatives guttiferone A, guttiferone E, and clusianone could inhibit the acetyltransferase activity of p300/CBP while another such compound nemorosone could activate the enzyme. Ravindra et. al. showed in his publication in 2009 (Journal of Biological Chemistry (2009), 284(36), 24453-24464) that naturally occurring hydroxynapthoquinone, plumbagin could inhibit p300 acetyltransferase activity. Arif et. al. in his publication in 2009 (Journal of Medicinal Chemistry (2009), 52(2), 267-277) discussed in detail the possible mechanisms by which garcinol, isogarcinol and LTK-14 could inhibit the acetyltransferase activity of p300. Yamaguchi et. al. disclosed in his patent JP 2000044468 A 20000215 that garcinol could inhibit lipase activity and be used for anti-obesity treatment. Bharate et. al. disclosed in his patent WO 2016063296 A1 20160428 that certain polyprenylated phloroglucinol compounds could have p-glycoprotein inducing activity and could therefore find application in treatment of Alzheimer's disease.

The prior art references mainly focus on targeting p300 acetyltransferase activity and some of the compounds having anti-adipogenic effects have been found to exert non-specific action on several other enzymes. The compound disclosed in this present invention has the most unique property of specifically targeting a new activity of p300 i.e. its butyryltransferase activity. Previously there has been no such work illustrating the importance of p300 catalysed butyrylation in the context of adipogenesis and obesity. Due to the fact that the compound disclosed in the present invention can inhibit only the butyryltransferase activity of p300, the acetylatransferase activity of p300 remains uninhibited—a property that has not been shown in the previously mentioned molecules. Besides specificity, the compound disclosed in the present invention has also been found to have no toxic effects on the cell line used for the experiment. Moreover, the anti-adipogenic efficacy of the compound has been proved not only in cell line models but also in two different mice models indicating very high potency of the compound as an anti-obesity molecule with high specificity and negligible toxicity.

### OBJECTS OF THE INVENTION

Main object of the present invention is to provide a small molecule modulator that can specifically inhibit butyryl transferase activity of p300 without affecting its acetyltransferase activity.

Another object of the invention is to provide a small molecule modulator that can suppress adipogenesis by inhibiting p300 catalysed butyrylation, thereby highlighting the significance of this modification in the context of adipogenesis and obesity.

Further object of the invention is to provide a small molecule modulator that can arrest obesity, prevent adipose tissue hypertrophy and liver steatosis by inhibiting butyrylation without affecting acetylation and with minimal off-target effect.

Yet another object of the invention is to provide a pharmaceutically acceptable composition bearing compound of Formula I.

Another object of the invention is to provide a pharmaceutical composition comprising compound of Formula I which may be administered orally intended for lymphatic absorption for the regulation of adipogenesis and obesity.

### SUMMARY OF THE INVENTION

Accordingly, the present invention provides a substituted benzophenone compound having selective p300 catalysed histone butyrylation inhibitory activity of Formula I

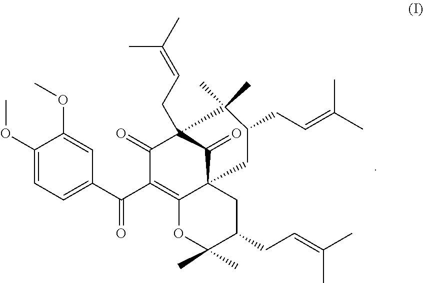

In an embodiment of the present invention the compound is useful as an anti-obesity agent by inhibiting p300 catalysed histone butyrylation without affecting acetylation and thereby specifically inhibiting adipogenesis without toxicity based side effects.

The present invention provides a process for preparation of the compound of Formula I as claimed in claim 1, comprising the steps of:
a) isolation of garcinol from Garcinia indica and synthesis of isogarcinol;
b) adding anhydrous potassium carbonate to a stirred solution of isogarcinol in dry acetone followed by dropwise addition of dimethyl sulfate under nitrogen gas atmosphere;
c) evaporating acetone and acidifying the reaction mass to obtain precipitate;
d) filtering and washing the precipitate and subjecting to chromatography on silica gel using eluting solvent mixture and
e) isolating the Compound of Formula I as white solid.

In an embodiment of the present invention the eluting solvent mixture is 5-6% ethyl acetate in hexane.

The present invention provides a pharmaceutical composition for oral administration comprising,
(i) the compound of Formula I,

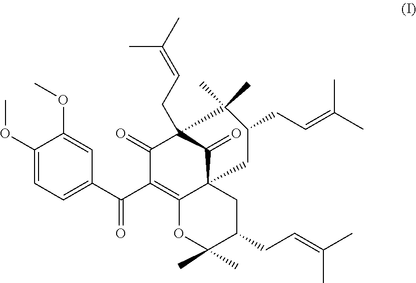

and
(ii) pharmaceutically acceptable excipient,
   wherein, the pharmaceutically acceptable excipient is selected from a group consisting of a lipid, a non-ionic surfactant, and a polymer and wherein the ratio of the compound of Formula I to excipient is 1:2.

In a preferred embodiment of the present invention the amount of the lipid is 0.15 to 0.4% w/v, the amount of non-ionic surfactant is 0.05 to 0.3% w/v and the amount of polymer is 0.05 to 0.25% w/v of the pharmaceutical composition.

In a preferred embodiment of the present invention the lipid is selected from the group consisting of monoglycerides, diglycerides, triglycerides, phosphatidylcholine, di-stearoyl phosphatidylcholine, di-stearoyl phosphatidylglycerol, and cholesterol.

In a preferred embodiment of the present invention the non-ionic surfactant is selected from the group consisting of poloxamers, cremophor and polysorbates.

In a preferred embodiment of the present invention the polymer is a pluronic polymer.

In an embodiment of the present invention the composition is a lipid based oral formulation.

In an embodiment of the present invention oral route is the preferential route of administration of the lipid based oral formulation to enhance oral bioavailability and to overcome solubility and permeability issues.

The formulation has potent and selective p300 catalysed histone butyrylation inhibitory activity.

The present invention provides a compound of formula 1,

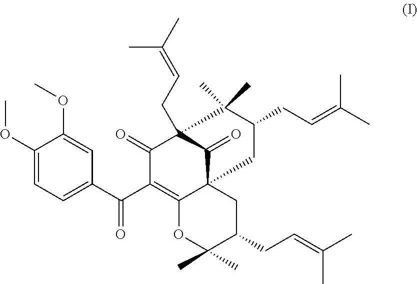

for use as a medicament for inhibiting adipogenesis.

The present invention provides a method of inhibiting adipogenesis by administering a therapeutically effective amount of a compound of Formula I,

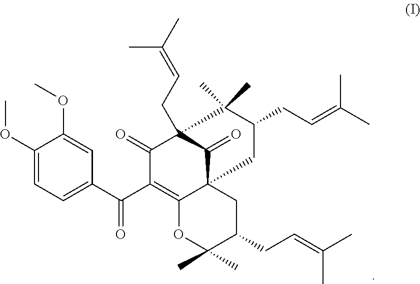

In an embodiment of the present invention the compound of general Formula I controls adipogenesis and control obesity.

In another embodiment of the invention wherein the compound of Formula I specifically inhibits the rarer and slower modifications catalysed by p300 and controls adipogenesis without too much toxicity based side effects.

In an another embodiment of the invention the compound of Formula I has significant physiological and metabolic stability.

In an embodiment of the present invention the composition is a lipid based oral formulation.

In an embodiment of the present invention oral route is the preferential route of administration of the lipid based oral formulation to enhance oral bioavailability and to overcome solubility and permeability issues.

### BRIEF DESCRIPTION OF THE DRAWINGS

The present invention may be more clearly understood by reference to the following Figures:

FIG. 1 illustrates the histone butyrylation marks increasing upon adipogenesis of 3T3L1 cells, in accordance with an embodiment of the present disclosure.

FIG. 2 illustrates the identification of compound of Formula I as an inhibitor of p300 catalyzed histone butyrylation that does not affect acetylation. It also highlights the non-specificity of garcinol, isogarcinol and some of its derivatives eg.—LTK-13, LTK-14 which can inhibit both p300 catalysed acetylation and butyrylation, in accordance with an embodiment of the present disclosure.

FIG. 3 illustrates the predicted differential interaction between different derivatives of garcinol with p300 catalytic domain. FIG. 3A is a schematic diagram of molecular docking of LTK-14 (a non-specific inhibitor of p300 catalysed acetylation and butyrylation) with the p300 catalytic domain. FIG. 3B is a schematic diagram of molecular docking of compound of Formula I (a specific inhibitor of p300 catalysed butyrylation) with the p300 catalytic domain, in accordance with an embodiment of the present disclosure.

FIG. 4 illustrates the toxicity study of compound of Formula I in 3T3L1 cell line by MTT assay, in accordance with an embodiment of the present disclosure.

FIG. 5 illustrates the inhibition of adipogenesis in 3T3L1 by compound of Formula I, as observed by Oil-Red O staining, in accordance with an embodiment of the present disclosure.

FIG. 6 illustrates the downregulation of pro-adipogenic gene expression by compound of Formula I in 3T3L1 cells, in accordance with an embodiment of the present disclosure.

FIG. 7 illustrates the histone modification status (downregulation of H4K5 and H3K23 butyrylation marks) in 3T3L1 cells upon treatment with compound of Formula I, in accordance with an embodiment of the present disclosure.

FIG. 8 illustrates the standardization of dosage and administration route of compound of Formula I for mice experiments, in accordance with an embodiment of the present disclosure.

FIG. 9 illustrates the obesity arresting effect of compound of Formula I in mice maintained on high fat diet, in accordance with an embodiment of the present disclosure.

FIG. 10 illustrates the histone butyrylation inhibition in liver and adipose tissue in high fat diet fed mice by compound of Formula I without affecting acetylation, in accordance with an embodiment of the present disclosure.

FIG. 11 illustrates the ability of compound of Formula I to reduce the weight of genetically obese leptin receptor mutant mice, in accordance with an embodiment of the present disclosure.

FIG. 12 illustrates the effect of compound of Formula I on the major organs involved in lipid metabolism i.e. liver and adipose tissue in genetically obese leptin receptor mutant mice, in accordance with an embodiment of the present disclosure.

FIG. 13 illustrates the histone butyrylation inhibition in liver and adipose tissue in genetically obese leptin receptor mutant mice by compound of Formula I without affecting acetylation, in accordance with an embodiment of the present disclosure.

FIG. 14 illustrates the bio-availability of compound of Formula I in mice due to the detection of its intact form in the liver, in accordance with an embodiment of the present disclosure.

FIG. 15 illustrates the bio-distribution properties of formulation containing compound of Formula I compared to free compound of Formula I administered at a concentration of 25 mg/Kg in SD rats, in accordance with an embodiment of the present disclosure.

FIG. 16 illustrates the bio-distribution properties of free compound of Formula I administered at concentrations of 25, 50, 100 and 200 mg/Kg in SD rats, in accordance with an embodiment of the present disclosure.

### DETAILED DESCRIPTION OF THE INVENTION

For convenience, before further description of the present disclosure, certain terms employed in the specification, and examples are delineated here. These definitions should be read in the light of the remainder of the disclosure and understood as by a person of skill in the art. The terms used herein have the meanings recognized and known to those of skill in the art, however, for convenience and completeness, particular terms and their meanings are set forth below. The invention will now be described in detail in connection with certain preferred and optional embodiments, so that various aspects thereof may be more fully understood and appreciated.

The articles “a”, “an” and “the” are used to refer to one or to more than one (i.e., to at least one) of the grammatical object of the article.

The terms “comprise” and “comprising” are used in the inclusive, open sense, meaning that additional elements may be included. It is not intended to be construed as “consists of only”.

The terms “comprise” and “comprising” are used in the inclusive, open sense, meaning that additional elements may be included. It is not intended to be construed as “consists of only”. Throughout this specification, unless the context requires otherwise the word “comprise”, and variations such as “comprises” and “comprising”, will be understood to imply the inclusion of a stated element or step or group of element or steps but not the exclusion of any other element or step or group of element or steps.

Ratios, concentrations, amounts, and other numerical data may be presented herein in a range format. It is to be understood that such range format is used merely for convenience and brevity and should be interpreted flexibly to include not only the numerical values explicitly recited as the limits of the range, but also to include all the individual numerical values or sub-ranges encompassed within that range as if each numerical value and sub-range is explicitly recited. For example, a temperature in the range of 60-120° C. should be interpreted to include not only the explicitly recited limits of 60° C.-120° C. but also to include sub-ranges, such as 61-119° C., and so forth, as well as individual amounts, within the specified ranges, such as 65.2° C., and 110.5° C.

The present invention relates to a polyisoprenylated benzophenone derivative compound of Formula I which selectively targets p300 catalysed butyrylation but not acetylation

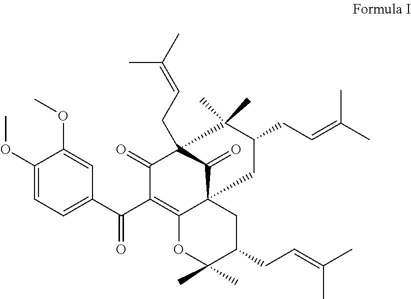

The compounds that fall under the definition of Formula I has the ability to inhibit adipogenesis in cell line model system without exerting any toxic effects. Moreover, histone butyrylation was found to be inhibited while acetylation remained unperturbed, indicating the selective nature of the polyisoprenylated benzophenone compounds in cell lines. Further studies have been conducted in two mice models of obesity-high fat diet induced obese mice and leptin receptor mutation carrying genetically obese mice. In both cases, administration of compound of Formula I helped in arresting obesity, preventing adipose tissue hypertrophy and liver steatosis. Total adiposity of the mice was reduced with minimal effect on lean mass, indicating that the compound did not have off-target side effects on animal health. Mechanistically, the compound of Formula I was found to have inhibited site-specific butyrylation in the liver of the mice, thereby inhibiting lipogenesis. Acetylation levels of histones were relatively unaffected implying specificity of the compound in animal models as well.

In an embodiment of the present disclosure, there is provided a compound of Formula (I)

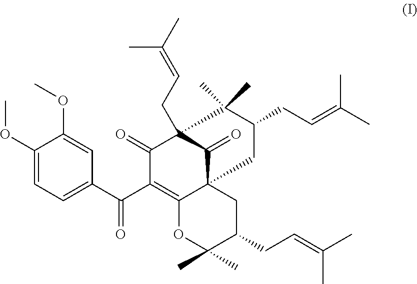

In an embodiment of the present disclosure, there is provided a compound of Formula I as disclosed herein, wherein the compound is useful as an anti-obesity agent by inhibiting p300 catalysed histone butyrylation without affecting acetylation and thereby specifically inhibiting adipogenesis without toxicity based side effects.

In an embodiment of the present disclosure, there is provided a process for preparation of the compound of Formula I as disclosed herein, comprising the steps of:
a) isolation of garcinol from Garcinia indica and synthesis of isogarcinol;
b) adding anhydrous potassium carbonate to a stirred solution of isogarcinol in dry acetone followed by dropwise addition of dimethyl sulfate under nitrogen gas atmosphere to obtain a reaction mass;
c) evaporating acetone and acidifying the reaction mass to obtain a precipitate;
d) filtering and washing the precipitate and subjecting to chromatography on silica gel using eluting solvent mixture; and
e) isolating the compound of Formula I as white solid.

In an embodiment of the present disclosure, there is provided a process for preparation of the compound of Formula I as disclosed herein, wherein the eluting solvent mixture is 5-6% ethyl acetate in hexane.

In an embodiment of the present disclosure, there is provided a pharmaceutical composition for oral administration comprising,
(i) the compound of Formula I,

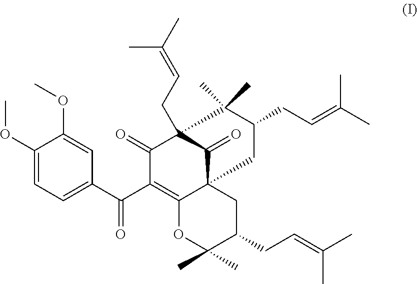

and
(ii) a pharmaceutically acceptable excipient,
   wherein, the pharmaceutically acceptable excipient is selected from a group consisting of a lipid, a non-ionic surfactant, and a polymer; and the ratio of compound of Formula I to the excipient is 1:2.

In an embodiment of the present disclosure, there is provided a pharmaceutical composition comprising the compound of Formula I and a pharmaceutically acceptable excipient selected from a group consisting of a lipid, a non-ionic surfactant, and a polymer, wherein the amount of the lipid is 0.15 to 0.4% w/v, the amount of the non-ionic surfactant is 0.05 to 0.3% w/v, the amount of the polymer is 0.05 to 0.25% w/v of the pharmaceutical composition, and the ratio of compound of Formula I to the excipient is 1:2.

In an embodiment of the present disclosure, there is provided a pharmaceutical composition as disclosed herein, wherein the lipid is selected from a group consisting of monoglycerides, diglycerides, triglycerides, phosphatidylcholine, di-stearoyl phosphatidylcholine, di-stearoyl phosphatidylglycerol, and cholesterol.

In an embodiment of the present disclosure, there is provided a pharmaceutical composition as disclosed herein, wherein the non-ionic surfactant is selected from a group consisting of poloxamers, cremophor, and polysorbates.

In an embodiment of the present disclosure, there is provided a pharmaceutical composition as disclosed herein, wherein the polymer is a pluronic polymer.

In an embodiment of the present disclosure, there is provided a pharmaceutical composition comprising the compound of Formula I and a pharmaceutically acceptable excipient selected from a group consisting of a lipid selected from a group consisting of monoglycerides, diglycerides, triglycerides, phosphatidylcholine, di-stearoyl phosphatidylcholine, di-stearoyl phosphatidylglycerol, and cholesterol, a non-ionic surfactant selected from a group consisting of poloxamers, cremophor, and polysorbates, and a pluronic polymer, wherein the amount of the lipid is 0.15 to 0.4% w/v, the amount of the non-ionic surfactant is 0.05 to 0.3% w/v, the amount of the polymer is 0.05 to 0.25% w/v of the pharmaceutical composition, and the ratio of compound of Formula I to the excipient is 1:2.

In an embodiment of the present disclosure, there is provided a compound of Formula (I),

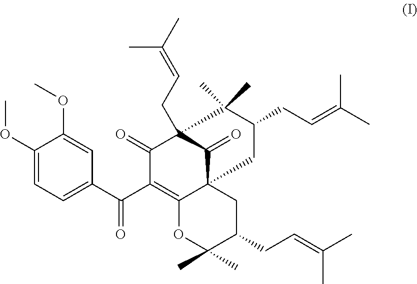

for use as a medicament for inhibiting adipogenesis.

In an embodiment of the present disclosure, there is provided a method of inhibiting adipogenesis by administering a therapeutically effective amount of a compound of Formula I,

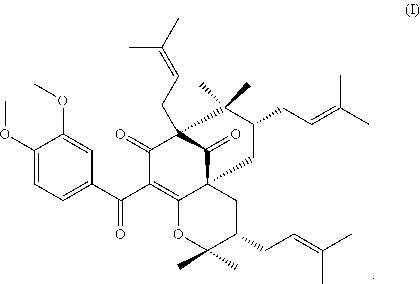

Although the subject matter has been described with reference to specific embodiments, this description is not meant to be construed in a limiting sense. Various modifications of the disclosed embodiments, as well as alternate embodiments of the subject matter, will become apparent to persons skilled in the art upon reference to the description of the subject matter. It is therefore contemplated that such modifications can be made without departing from the spirit or scope of the present subject matter as defined.

### EXAMPLES

The disclosure will now be illustrated with the working examples, which is intended to illustrate the working of disclosure and not intended to take restrictively to imply any limitations on the scope of the present disclosure. Unless defined otherwise, all technical and scientific terms used herein have the same meaning as commonly understood to one ordinary person skilled in the art to which this disclosure belongs. Although methods and materials similar or equivalent to those described herein can be used in the practice of the disclosed methods and compositions, the exemplary methods, devices, and materials are described herein. It is to be understood that this disclosure is not limited to particular methods, and experimental conditions described, as such methods and conditions may apply.

### Abbreviations

H3K23—Histone 3 Lysine 23
H4K5—Histone 4 Lysine 5

### Synthesis: General Procedure for the Synthesis of Compound of Formula I:

Isolation of garcinol from Garcinia indica and synthesis of isogarcinol from it were carried out following the protocols as mentioned in Mantelingu et al, (2007) Chem Biol. For synthesis of compound of Formula I, anhydrous potassium carbonate was added to a stirred solution of isogarcinol in dry acetone followed by dropwise addition of dimethyl sulfate under nitrogen gas atmosphere. Acetone was evaporated from the reaction mass which was then acidified with 5% aqueous HCl. The precipitate was filtered, washed, and chromatographed on silica gel using 5-6% ethyl acetate in hexane as eluting solvent mixture. Compound of Formula I was isolated as white solid, and NMR spectral analysis was consistent with the desired product.

The NMR spectra details are as follows—¹H NMR (400 MHZ, DMSO-d6): δH 7.29 (d, J=1.9 Hz, 1H, Ar—H), 7.10 (dd, J=8.5, 1.9 Hz, 1H, Ar—H), 6.97 (d, J=8.5 Hz, 1H, Ar—H), 5.14, 4.89, 4.77 (brt, ¹H each), 3.80, 3.79 (s, 3H each, OCH3), 2.87-1.29 (m, 12H, methylene and methyne), 1.74, 1.66, 1.62, 1.59, 1.53, 1.51, 1.21.1.06, 0.91, 0.83 (s, 3H each, methyl); HR-ESIMS (m/z): found 631.3990 [M+H]+, calc. 631.3999.

The purity of the synthesized compound x (Formula I) was verified by LC-MS analysis whereby a single peak was found in the UV chromatogram of the sample indicating sample homogeneity and only the protonated species of compound of Formula I were observed in the ESI spectra of the corresponding fraction.

Following examples are given by way of illustration and should not construe the scope of the present invention.

Garcinia indica fruit rinds were obtained from the Brindonia Tallow tree or Garcinia indica (Thouars) Choisy (Family: Clusiaceae) in the Western Ghats, India.

### Example 1 (Isolation of Garcinol)

Garcinia indica fruit rinds dried powder (1 kg) was extracted with methanol (5 L×3) in a percolator for three consecutive days (24 h×3). The combined methanolic extract was filtered through whatman filter paper (G-I grade) and concentrated on rotavapor under reduced pressure to give brown colored viscous residue (618 g). The residue was dissolved in water (1.5 L) and extracted with ethyl acetate (1 L×3); the combined organic layer was washed with brine solution, dried over anhydrous sodium sulfate, and evaporated under vacuum to obtain crude mass (190 g). The crude mass was purified by silica gel column chromatography using 8-10% ethyl acetate:hexane as eluting solvent to obtain fractions containing garcinol. Garcinol (10 g) was obtained in pure form by recrystallization in hexane.

### Example 2 Synthesis of Isogarcinol

Garcinol (3 g) in toluene was stirred in round bottom flask. Conc HCl (1.5 ml) was added drop wise to the solution and was kept under stirring for 24 h at 40° C. Toluene was removed under vacuum and crushed ice was added to the reaction mass. Heavy precipitated was observed, it was filtered on sintered funnel and several washing with cold water was given to obtain pale white solid which was further re-crystallized from acetonitrile to afford pure isogarcinol (2.12 g).

### Synthesis of Polyisoprenylated Benzophenone Compound of Formula I Example 3

Isogarcinol (100 mg) and anhydrous potassium carbonate (56 mg) were stirred in dry acetone (10 ml). After 1 h, dimethyl sulfate (52 mg) was added slowly under nitrogen atmosphere and stirring was continued for 14 h. On completion of reaction as checked by TLC, acetone was removed under vacuum. Crushed ice was added to the crude mass thus obtained and further acidified with 5% HCl (pH 5-6). A pale brown precipitate was observed; it was filtered and washed with cold water. The dried solid was purified by column chromatography on silica gel using 5-6% ethyl acetate:hexane solvent system to obtain white solid compound of Formula I (80 mg).

### Example 4

A solution of isogarcinol (200 mg) in acetone (20 ml) was stirred for 30 minutes at 20-25° C. followed by the addition of potassium carbonate (91.7 mg) and methyl iodide (117.6 mg). The reaction mixture was stirred for 10-12 h at 20-25° C. The progress of the reaction was monitored by TLC analysis. After stirring the reaction mixture for 10-12 h, the acetone was completely distilled off and quenched in 10 ml of water. The product was extracted with ethyl acetate, washed with water, and concentrated. Finally, compound of Formula I was obtained in pure form by column chromatography using silica gel (230-400 mesh) and ethyl acetate in hexane solvent system.

### Example 5

To a solution of isogarcinol (4 g) in dry acetone, anhydrous potassium carbonate (2.4 g) was added. The solution was stirred for 30 minutes then dimethyl sulfate (2.2 g) was added drop wise under nitrogen atmosphere and stirring was continued for further 14 h. After removing acetone, crushed ice was added and acidified with 5% HCl (pH 5-6). It was extracted with ethyl acetate (200 ml×3), dried over anhyd. Na₂SO₄ and concentrated to obtain yellow viscous residue (5 g). The residue was subjected to column chromatography on silica gel using 5-6% ethyl acetate:hexane solvent system to obtain white solid compound of Formula I (2.7 g).

### Biological Studies: Example 6: Adipogenesis of 3T3L1 Cell Line

The preadipocyte cell line 3T3L1 was seeded in complete DMEM (supplemented with 10% FBS). Once the cells reached almost full confluent state, they were treated with differentiation media (2 μg/mL Insulin, 0.5 mM IBMX and 1 μM Dexamethasone) for 2 days. After the differentiation induction, the cells were kept in maintenance media (2 μg/mL Insulin) for another 4-6 days with media change given every 2 days. The completion of adipogenesis was confirmed by the observation of lipid droplet accumulation in most of the cells. On the 6^{th} day post induction of differentiation the cells were fixed with 3.7% formaldehyde solution followed by staining with Oil Red-O dye (prepared in 60% isopropanol) for 1 hour at room temperature. The incorporated dye was extracted using isopropanol and absorbance values were determined spectrophotometrically at 510 nm to estimate total amount of lipid accumulation under different experimental conditions.

### Example 7: Acid Extraction of Histones and Immunoblotting for Post-Translational Modifications

Histones were acid extracted from both undifferentiated and differentiated 3T3L1 for comparison of acetylation and butyrylation levels. For the acid extraction, 3T3L1 cells were washed with ice-cold PBS (supplemented with 5 mm NaBu) and then resuspended in Triton extraction buffer (PBS containing 0.5% Triton X-100 (v/v) and 2 mM PMSF). The cells were allowed to lyse on ice for 10 minutes, spun down, washed with PBS and the process was repeated another time. Then the proteins were acid extracted with 0.2 N HCl for two hours on ice. The debris was spun down and then histones were precipitated by incubating the supernatant with 33% Trichloroacetic acid at 4° C. for 30 minutes. The histones were then pelleted down and then given two washes with acetone. The pellet was allowed to air-dry for 10 minutes and then resuspended with 50 mm Tris-Cl, pH 7.4.

The histones were then run in 15% SDS-PAGE, transferred onto PVDF membrane, blocked with 5% skimmed milk solution to prevent non-specific antibody binding, and finally probed with antibodies against acetylation and butyrylation marks.

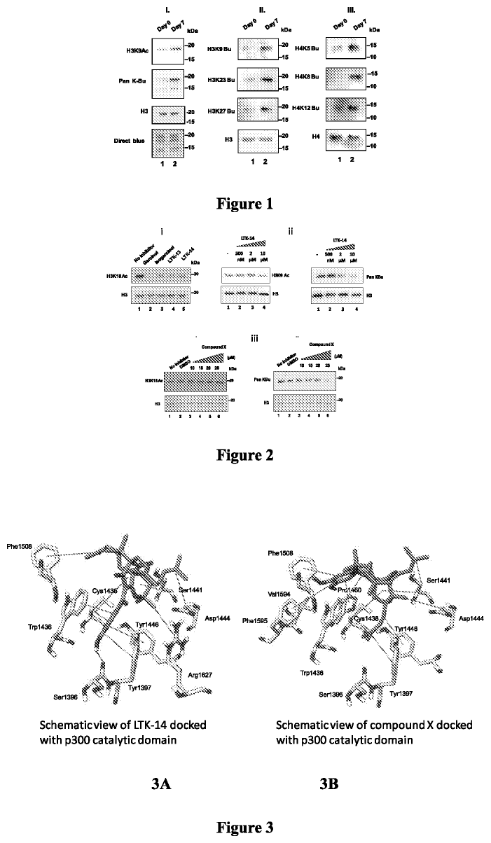

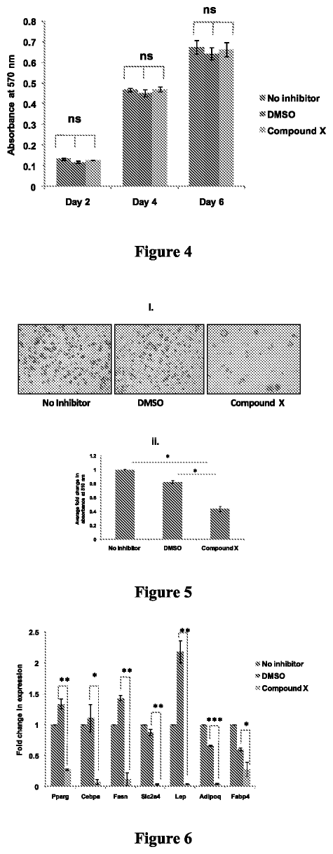

Histone acetylation and butyrylation levels in 3T3L1 cells in undifferentiated and differentiated state were analysed by immunoblotting with antibodies specific for acetylated H3K9, butyrylated H3K9, H3K23, H3K27, H4K5, H4K8, H4K12 and pan-butyryl lysine antibody which recognizes butyrylation on any site (FIG. 1). H3 and H4 were used as loading controls along with Direct Blue staining of all four core histones. Lane 1, undifferentiated preadipocyte (Day 0) and Lane 2, differentiated adipocyte (Day 7) in each of the panels i, ii and iii.

Besides increase in acetylation, as was known before, an increase in butyrylation was observed at several sites of histones upon adipogenesis.

### Example 8: In Vitro Acetylation/Butyrylation Assay for Screening of Small Molecule Modulators

Purified full length p300 protein was used for in vitro acetylation/butyrylation assay in presence/absence of different compounds. Acylation reactions were performed in reaction buffer (25 mM Tris-HCl PH 7.5, 100 mM NaCl, 0.1 mM EDTA, 1 mM DTT, 10% Glycerol, 1×PIC) with 100 ng/ml TSA, and 50 μM Butyryl-CoA/Acetyl COA. Xenopus histone H3 (500 ng) was used as the substrate and p300 (10,000 cpm activity) was used as the enzyme. Reactions were incubated in presence or absence of small molecule modulators (including compound of Formula I) for 10 minutes at 30° C., followed by initiation of the acetylation/butyrylation reaction by the addition of acetyl/butyryl CoA. After a further 10 minutes, reactions were stopped by addition of Laemmli buffer and samples were used for immunoblotting.

Immunoblotting with antibodies against acetylated H3K18, acetylated H3K9 and pan-butyryl lysine was performed to check for the relative inhibition of acetyl transferase and butyryl transferase activities of full length p300 by garcinol, isogarcinol and its different derivatives including compound of Formula I at different concentrations. FIG. 2i depicts the effect of garcinol, isogarcinol and two of its derivatives LTK-13 and LTK-14 on p300 catalysed acetyltransferase activity. All of these compounds showed inhibitory effect. FIG. 2ii depicts the effect of LTK-14 on acetylation and butyrylation catalysed by p300. Lane 1, enzyme alone; Lane 2-4, enzyme+LTK-14 at concentrations 500 nM, 2 μM and 10 μM, respectively. LTK-14 could inhibit acetylation at 10 μM concentration while butyrylation was inhibited at 2 μM concentration. FIG. 2iii depicts the effect of compound x on acetylation and butyrylation catalysed by p300. Lane 1, enzyme alone; Lane 2, enzyme+DMSO; Lane 3-6, enzyme+compound x at concentrations 10, 15, 20 and 25 μM, respectively. The compound of Formula I was found to have a significant inhibitory effect on butyrylation at 25 μM without affecting acetylation in the same range of concentration.

### Example 9: Molecular Docking Analysis

Molecular docking experiments were performed using Schrödinger was used for the preparation of ligands and proteins for docking. p300 catalytic domain (PDB: 3BIY) (Liu, X., et al., The structural basis of protein acetylation by the p300/CBP transcriptional coactivator, Nature 451, 846-850 (2008)) structural information was obtained from Protein Data Bank. The geometry was optimized in vacuo using the steepest descent followed by conjugate gradient algorithms. Schrödinger was used to generate a statistically significant number of docked poses. The results were clustered using standard deviation cut off of 3 Å. Docked structure was further solved using Pymol software.

It was observed (FIG. 3i) that the non-specific monosubstituted derivative of isogarcinol—LTK-14 may form a hydrogen bond with carbonyl oxygen atom in the peptide backbone of Serine 1396 of p300 catalytic domain active site with its only free phenolic —OH group. On the other hand in FIG. 3ii, the disubstituted derivative (compound of Formula I) cannot form such hydrogen bonds as both of its phenolic —OH groups are blocked. The difference in this interaction pattern could explain why LTK-14, by forming an additional critical hydrogen bond can inhibit both acetylation and butyrylation catalysed by p300. On the other hand, the compound of Formula I cannot form this interaction and therefore it can only inhibit the slower butyrylation reaction.

### Example 10: MTT Assay for Cytotoxicity Testing

12500 cells/mL were seeded in presence/absence of compound of Formula I in 90-well plate and at different intervals of time (2 days, 4 days, 6 days), 1/10^{th} volume of 5 mg/ml MTT solution was added to it followed by incubation at 37° C. for 3 hours. The formed crystals were dissolved in DMSO and spectrometric estimation of colour intensity was done at 570 nm.

MTT assay was performed to check for any possible cellular toxicity effect of compound of Formula I, using 25 μM concentration of the compound on 3T3L1 cells upon incubation for varying lengths of time (2, 4 and 6 days). Comparison was done with cells that were left untreated or treated with DMSO as solvent control (FIG. 3).

No toxic effect of compound of Formula I could be observed in 3T3L1 cells over the mentioned duration of experiment.

### Example 11: RNA Isolation for Gene Expression Analysis by Q-PCR

Total RNA was isolated from the treated 3T3L1 cells by phenol-chloroform method. The isolated RNA samples were treated with 2 units/μL DNase I to remove any residual DNA followed by overnight ethanol precipitation at −80° C. RNA integrity was checked in 1% agarose gel. 2 μg RNA was used for cDNA synthesis using oligo dT primers. RT-PCR was done using primers of adipogenesis related genes followed by detection with SYBR Green.

RT-PCR analysis was performed to show downregulation of genes associated with adipogenesis by compound of Formula I (FIG. 5). Error bars denote mean+/−SD of three biological replicates, one-way ANOVA with Dunnett's multiple comparison; * P<0.05, ** P<0.01, *** P<0.001, ns: not significant.

### Example 12: Immunoblotting to Check Histone Modification Status in 3T3L1 Upon Treatment with Compound of Formula I

Acid extraction of histones was performed from 3T3L1 cells that had been induced to undergo adipogenesis in the absence of any inhibitor (No Inhibitor), in presence of DMSO (solvent control) and in presence of compound of Formula I. The extraction protocol was the same as that described in Example 7.

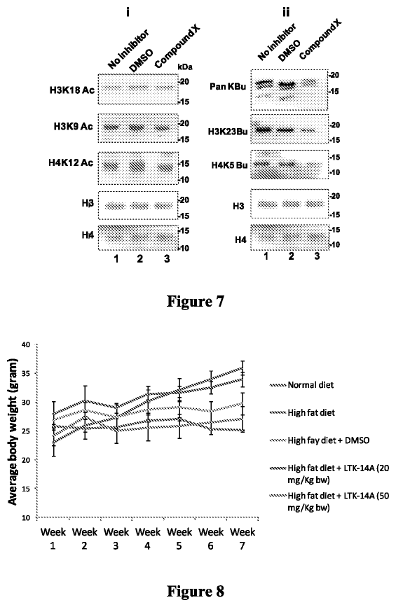

Histone acetylation levels in 3T3L1 cells that were treated with compound of Formula I were compared with that in DMSO treated and untreated conditions by immunoblotting with antibodies against acetylated histones H3K18, H3K9 and H4K12 (FIG. 7). Histones H3 and H4 were used as loading controls (panel i). Under similar conditions histone butyrylation levels were compared by immunoblotting using antibodies against butyrylated histone H3K23, H4K5 and pan-butyryl lysine (panel ii).

Acetylation was not found to be affected by compound x treatment. However, H4K5 Bu and H3K23 Bu marks showed inhibition upon compound x treatment, indicating that the compound x is a site-specific inhibitor of butyrylation that does not affect acetylation in 3T3L1.

### Example 13: Standardization of Dosage and Administration Mode of Compound of Formula I for Animal Experiment

Male C57BL/6J mice (8 weeks old) bred in house were obtained and acclimatized for 1 week prior to experiments. Mice were maintained on a 12-hour light/dark cycle at 22+/−3° C. and a relative humidity of 55% and given ad libitum access to food and water. The age-matched C57BL/6J mice were maintained on a standard laboratory chow diet or high fat diet (60 kcal % fat) and water. Two groups were intraperitoneally injected with compound x at two different doses (20 mg/Kg bw and 50 mg/kg bw) twice every week. A third group was injected with equivalent volume of DMSO (for 50 mg/kg bw) as vehicle control. Body weight and food intake was measured twice every week.

It was observed that a 20 mg/kg bw was not sufficient for arresting weight gain in the mice maintained on high fat diet (FIG. 8). However, 50 mg/kg bw did have an attenuating effect on body weight. An equivalent volume of DMSO injection led to an almost similar arrest in weight gain. Therefore it was not possible to infer whether compound x itself can prevent body weight gain of mice or not.

It was concluded that instead of intraperitoneal injection, the compound x would be mixed with the diet for painless administration inside the animals without any toxicity effect of vehicle. The dosage of 50 mg/Kg bw was finalized for the next experiment, based on the observation.

### Example 14: Effect of Compound of Formula I on Weight Gain of Mice Maintained on High Fat Diet

Male C57BL/6J mice (8 weeks old) were obtained and acclimatized for 1 week prior to experiments. Mice were maintained on a 12-hour light/dark cycle at 22+/−3° C. and a relative humidity of 55% and given ad libitum access to food and water. The age-matched C57BL/6J mice were maintained on a standard laboratory chow diet or high fat diet (60 kcal % fat) with or without compound of Formula I (50 mg/kg body weight) and water. Body weight and food intake was measured twice every week.

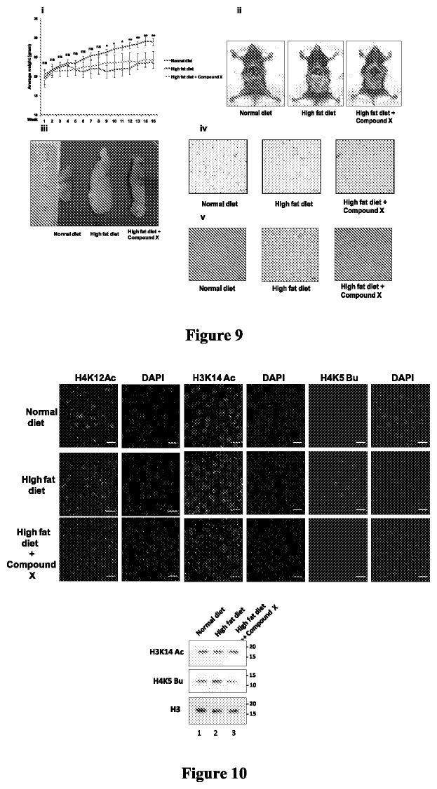

Trend of weight gaining after 16 weeks of C57BL6/J mice maintained on normal chow diet, high fat diet and high fat diet mixed with compound of Formula I was plotted (FIG. 9) (panel i); n=4 in each group:one-way ANOVA with Dunnett's multiple comparison; * P<0.05, ** P<0.01, *** P<0.001, ns: not significant. Representative images of dissected bodies showing fat accumulation (panel ii) and epididymal fat pads (panel iii) of the mice from three groups. Representative images of morphology of adipose tissue (iv) and liver (v) from the mice of the three groups, as studied by hematoxylin and eosin staining.

It was observed that compound of Formula I at a dosage of 50 mg/kg bw, when mixed with high fat diet, prevented weight gain of the mice compared to those maintained on high fat diet without the compound. Moreover, the liver also showed less lipid accumulation and adipose tissue exhibited less hypertrophy upon compound of Formula I treatment.

### Example 15: Immunohistochemistry and Immunoblotting for Checking the Effect of Compound of Formula I on Histone Modifications in the Liver and Adipose Tissue Respectively, of Mice Maintained on High Fat Diet

Collected liver tissue samples were stored in 4% paraformaldehyde for 24 hours after which they were cryoprotected in 30% sucrose solution for two weeks. Cryosections were performed at 7 μm sections using Cryostat Leica CM1850 UV. For staining, the tissue sections were washed with PBS followed by antigen retrieval with 0.01 M citrate buffer (pH 6). The tissues were then permeabilised with 0.3% Triton X-100/PBS (PBST) and blocked with 2% serum followed by incubation with primary antibodies overnight at 4° C. The next day, secondary antibody incubation was carried out for one hour at room temperature followed by staining of the nuclei with Hoechst 33342 and mounting with 70% glycerol. Images were acquired by confocal microscopy. For quantitation, intensity of each modification specific staining was normalized with respect to the Hoechst staining intensity.

It was observed that H4K5 butyrylation in liver increased upon high fat diet consumption but was inhibited in presence of compound of Formula I (FIG. 10A). On the other hand, H4K12 and H3K14 acetylation was not affected, indicating that compound of Formula I is specifically inhibiting butyrylation instead of acetylation. Acid extraction of histones was performed from the epididymal fat pads of mice using the same protocol as mentioned in example 7. It was observed that H4K5 butyrylation was reduced in the adipocytes of high fat diet fed mice treated with compound of Formula I compared to mice fed with high fat diet only (FIG. 10B).

### Example 16: Effect of Compound of Formula I on Body Weight Gain of Genetically Obese Mice

Male db+/− and genetically obese db/db mice (8 weeks old) were obtained and acclimatized for 1 week prior to experiments. Mice were maintained on a 12-hour light/dark cycle at 22+/−3° C. and a relative humidity of 55% and given ad libitum access to food and water.

The age-matched db+/− and db/db mice were maintained on a standard laboratory pelleted diet and water. db/db mice were oral gavaged (50 mg/kg) with LTK14A in 0.5% carboxymethyl cellulose, equal volume of the vehicle was orally gavaged to control db/db mice for a total period of 30 days. db+/− animals were kept as untreated normal control group. The body weights and feed intake was recorded every week throughout the experimental period. The lean and fat mass of the mice were determined using Echo MRI in live mice on day 15^{th} and day 30^{th}.

Trend of weight gaining after 30 days of genetically obese leptin receptor homozygous mutant mice with compound of Formula I was plotted against that of mutant mice treated with vehicle control and also heterozygous mutant mice (FIG. 10)(panel i). Average weight gain, average fat mass and average lean mass of the mice from the three groups are represented in bar p graphs (panel ii, iii and iv respectively); n=4 in each group:one-way ANOVA with Dunnett's multiple comparison; * P<0.05, ** P<0.01, *** P<0.001, ns: not significant.

It was observed that compound of Formula I treatment led to a reduction in body weight of genetically obese db/db mice compared to those treated with vehicle control. This was corroborated by Echo MRI results which showed that compound of Formula I had a greater effect on fat mass of the mice compared to lean mass.

### Example 17: Effect of Compound of Formula I on Liver and Adipose Tissue of Genetically Obese Mice

After the treatment of the mice with compound of Formula I for one month, the mice were sacrificed and their liver and epididymal fat pads were harvested. The organs were individually weighed and then processed for morphology study by hematoxylin and eosin staining.

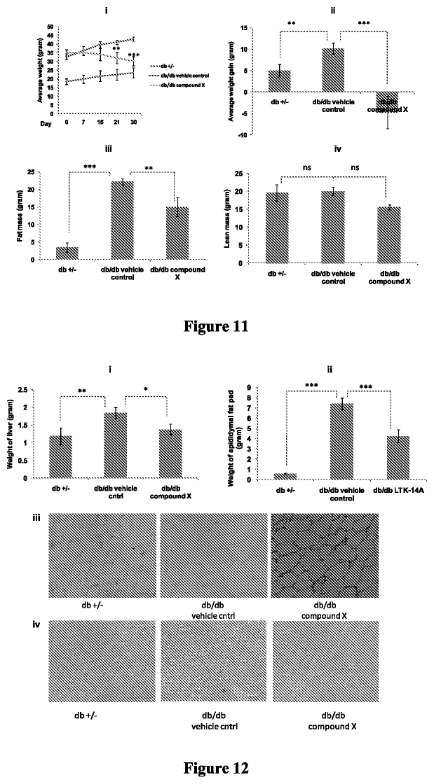

Average weights of liver (i) and adipose tissue (ii) of the mice of the three different groups are represented by bar graphs (FIG. 11); n=4 in each group:one-way ANOVA with Dunnett's multiple comparison; * P<0.05, ** P<0.01, *** P<0.001, ns: not significant. Representative images of morphology of adipose tissue (iii) and liver (iv) from the mice of the three groups, as studied by hematoxylin and eosin staining.

Both the liver and epididymal fat pads weighed the highest for db/db mice given vehicle control, while that for the db+/− mice was lowest. The db/db mice treated with compound x showed intermediate weights indicating that the compound x has the ability to reduce the weight of obese mice by impacting the physiology of the two organs central to lipid metabolism—the liver and adipose tissue. Moreover, while the obese mice with vehicle control treatment exhibited symptoms of steatosis in liver (appearance of ballooning hepatocytes) and adipose tissue hypertrophy, the symptoms were significantly reduced in mice treated with compound of Formula I.

### Example 18: Immunohistochemistry and Immunoblotting for Checking the Effect of Compound of Formula I on Histone Modifications in the Liver and Adipose Tissue Respectively, of Mice Maintained on High Fat Diet

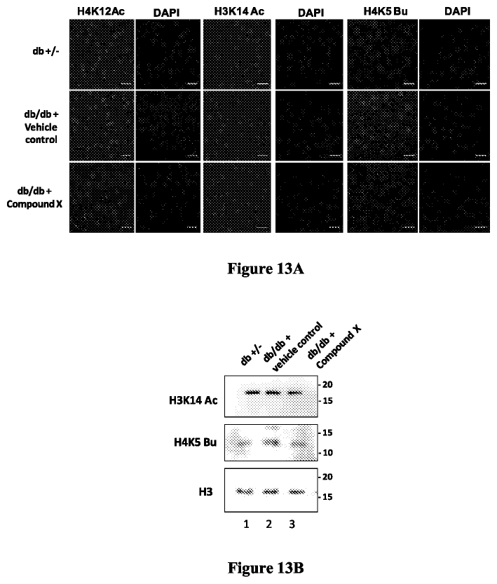

Liver sample sectioning and immunohistochemical staining was performed using the same protocol as mentioned in example 15. It was observed that H4K5 butyrylation in liver increased in the db/db obese mice liver but was inhibited in presence of compound of Formula I (FIG. 13A). On the other hand, H4K12 and H3K14 acetylation was not affected, indicating that compound of Formula I is specifically inhibiting butyrylation instead of acetylation. Acid extraction of histones was performed from the epididymal fat pads of mice using the same protocol as mentioned in example 7. It was observed that H4K5 butyrylation was reduced in the adipocytes of db/db mice treated with compound of Formula I compared to db/db mice treated with vehicle control (FIG. 13B).

### Example 19: Untargeted Analysis of Intracellular Metabolites in Mice Liver for Detection of Compound of Formula I by Ultra-Performance Liquid Chromatography Coupled with Time-of-Flight Mass Spectrometer (Q-TOF LC/MS)

The liver samples of mice treated with compound I were homogenized in a solution consisting of 50% methanol and 50% water and then snap frozen in liquid nitrogen followed by lyophilization. Profiling of intracellular metabolites was performed on an agilent 1290 Infinity LC system coupled to Agilent 6545 Accurate Mass Quadrupole Time of Flight (QTOF) with Agilent Jet Stream Thermal Gradient Technology. The UPLC system was assembled with a Diode array detector (DAD) and autosampler. The Chromatographic separation was achieved on Agilent ZORBAX SB-C18 column (2.1×100 mm, 1.8 μm) as stationary phase. For untargeted analysis, the mobile phase consisted of a linear gradient of 100 mM ammonium formate (A) and Acetonitrile (B): 0-10.0 min, 30-80% B (v/v); 10.0-15.0 min, 80-100% B (v/v); 15.0-20.0 min, 100% B (v/v); 20.0-21.0 min, 100-30% B (v/v); 21.0-25.0 min, 30% B. For targeted analysis, the mobile phase consisted of a linear gradient of 100 mM ammonium formate (A) and Acetonitrile (B): 0-5.0 min, 0-50% B (v/v); 5.0-6.5 min, 50-100% B (v/v); 6.5-8.0 min, 100% B (v/v); 8.0-9.0 min, 100-0% B (v/v); 9.0-15.0 min, 100% A. The sample was dissolved in 1 mL methanol (LCMS Grade), centrifuged and supernatant was taken for UPLC-QTOF-MS analysis. The column was reconditioned for 5 minutes prior to the next injection. The flow rate was 0.5 mL/min, and the injected volume was 20 μL. The UPLC was connected to the MS analysis was performed on an Agilent 6545 Accurate-Mass Q-TOF/MS system with an electrospray ionization (ESI) source. Considering the MS conditions, positive ion mode was used to obtain high-resolution mass spectra. The ESI source parameters were: drying gas (N2) flow, 8 L/min; drying gas temperature, 200° ° C. Other parameters were set as nebuliser gas, 35 psig; capillary voltage, 3000 V; skimmer voltage, 65 V; nozzle voltage 1000 V and fragment or voltage 150 V. The data acquisition on the LC-QTOF was performed using Agilent Mass Hunter Acquisition software (Agilent Technologies, Santa Clara, CA, USA). The data were deconvoluted into individual chemical peaks with Agilent Mass Hunter Qualitative Analysis (Mass Hunter Qual, Agilent Technologies).

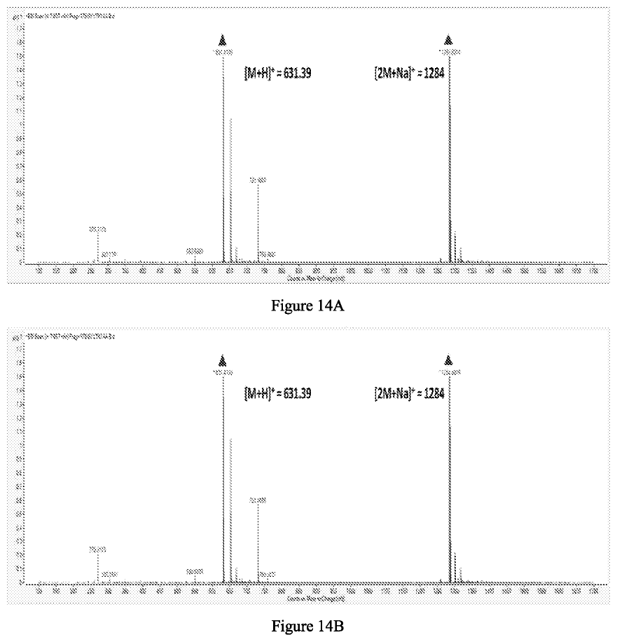

The ESI spectra of pure solution of compound of Formula I was taken as reference (FIG. 14A). When LC-ESI-MS analysis of metabolites of compound treated mice, liver was performed, the corresponding protonated molecular ion species was detected in the ESI spectra (FIG. 14B). This highlighted the bioavailability of compound of Formula I because it could be detected in its intact form in the mice liver.

### Example 20: Single Dose Toxicity Study of Compound X for Maximum Tolerated Dose

The single dose toxicity study on compound X was conducted in accordance with Good Laboratory Practice Principles as published by the OECD in 1998, in accordance with the Drugs and Cosmetics Rule (New Drugs and Clinical trials, 2019/DCGI). Compound X (formulated in vehicle i.e. 0.5% sodium salt of carboxymethyl cellulose) was administered to Sprague Dawley rats once by oral route on the day of scheduled treatment. The three treatment groups were 250 mg, 500 mg, and 1000 mg/kg body weight, while vehicle control was administered to control group. The rat equivalent doses were calculated from the dosage used in mice experiments following FDA guidelines for drug administration. The doses finally administered were 10, 20 and 40 times the corresponding mice equivalent dose. Food, but not water, was removed thus letting the animals fast over-night prior to dosing. After administration of LTK-14A, food was withheld for further 2-3 hours and the animals were observed at 30 minutes, 1 hour, 2 hours and 4 hours from the time of dosing. Animals were subsequently observed once daily for any signs of toxicity, mortality, or any other observations including conditions of skin, hair coat, eyes, mucous membrane, movement, activity, tremors, convulsions, salivation, diarrhea, posture, piloerection and lethargy. Food intake, water intake and body weight of animals was recorded weekly. Terminal sacrifice of surviving animals was performed after 14 days using CO2 euthanasia technique following which animals were necropsied. Animals were fasted over night before necropsy. Gross observations were recorded.

The body weight changes in the compound X administered rats were similar to the vehicle control treated ones (Table 1) with no gross morphological changes in any organ. Moreover, there was also no significant treatment related effect on the food and water consumption of rats of both gender for all three doses compared to vehicle control (Table 2 and 3).

TABLE 1
Details of single dose toxicity test Body weight (gram,
mean +/− SD of animals in all groups)

| | Male | | | | Female | | | |
|---|---|---|---|---|---|---|---|---|
| Group | | Initial | Mid | Final | | Initial | Mid | Final |
| Vehicle control | Mean | 174.75 | 217.38 | 232.13 | Mean | 152.21 | 169.81 | 171.5 |
| | ±SD | 7.61 | 11.38 | 18.38 | ±SD | 6.58 | 6.57 | 6.65 |
| Compound X (250 | Mean | 171.94 | 216.62 | 244.21 | Mean | 151.99 | 174.54 | 176.35 |
| mg/Kg bw) | ±SD | 6.87 | 13.63 | 29.81 | ±SD | 6.43 | 6.06 | 9.5 |
| Compound X (500 | Mean | 176.3 | 229.75 | 243.14 | Mean | 153.32 | 176.55 | 175.8 |
| mg/Kg bw) | ±SD | 5.53 | 17.08 | 12.25 | ±SD | 6.44 | 10.86 | 6.48 |
| Compound X (1000 | Mean | 176.24 | 217.65 | 226.19 | Mean | 153.81 | 178.58 | 179.52 |
| mg/Kg bw) | ±SD | 7.51 | 12.31 | 23.57 | ±SD | 7.29 | 7.46 | 8.45 |

TABLE 2
Average food intake (gram/day/animal) in each group

| | Male | | | Female | | |
|---|---|---|---|---|---|---|
| Group | Initial | Middle | Final | Initial | Middle | Final |
| Vehicle control | 20.38 | 20.24 | 21.24 | 17.84 | 15.65 | 15.95 |
| Compound X (250 | 20.03 | 20.79 | 22.62 | 20 | 16.32 | 16.65 |
| mg/Kg bw) | | | | | | |
| Compound X (500 | 20.99 | 21.14 | 23.29 | 18.22 | 18.13 | 17.09 |
| mg/Kg bw) | | | | | | |
| Compound X (1000 | 20.76 | 20.74 | 19.95 | 18.17 | 17.82 | 16.97 |
| mg/Kg bw) | | | | | | |

TABLE 3
Average water intake (ml/day/animal) in each group

| | Male | | | Female | | |
|---|---|---|---|---|---|---|
| Group | Initial | Middle | Final | Initial | Middle | Final |
| Vehicle control | 20.38 | 20.24 | 21.24 | 17.84 | 15.65 | 15.95 |
| Compound X (250 | 20.03 | 20.79 | 22.62 | 20 | 16.32 | 16.65 |
| mg/Kg bw) | | | | | | |
| Compound X (500 | 20.99 | 21.14 | 23.29 | 18.22 | 18.13 | 17.09 |
| mg/Kg bw) | | | | | | |
| Compound X (1000 | 20.76 | 20.74 | 19.95 | 18.17 | 17.82 | 16.97 |
| mg/Kg bw) | | | | | | |

### Example 21: Pharmacokinetics of Compound X Loaded Nanostructured Lipid Carrier

The lipid based formulations are reported to be promising approach that has ability to fix the issues related to absorption and metabolism. The oral route is the preferential route of administration therefore a lipid based oral formulation bearing compound of Formula I has been developed to enhance oral bioavailability and to overcome solubility and permeability issues. The compound of Formula I is small molecule that can arrest obesity, prevent adipose tissue hypertrophy and liver steatosis by inhibiting butyrylation without affecting acetylation. Attempt has been made to deliver molecule of Formula I through lymphatic route to overcome metabolism through cytochrome P450 (CYP) enzymes to avoid first pass elimination and poor in vivo absorption. The excipients belonging to GRAS (Generally Regarded As Safe) category shall be used.

A number of formulations were prepared with compound X for pharmacokinetic studies. The following are the types of formulations prepared.

### Example 22

The nanostructured lipid carrier was prepared by hot homogenization followed by cold ultrasonication. The liquid phase consisting of weighed quantities of Monostearin (0.3% W/V), Capryol90 (0.15% W/V) and compound X (0.05% W/V) were heated to 90° ° C. with stirring. The aqueous phase consisting of Pluronic F68 (0.1% W/V) was heated to the same temperature and subsequently added to lipid phase and mixed using a homogenizer set at 13000 rpm for 5 min. The resulting coarse emulsion was ultrasonicated in an ice bath for 5 min. The suspension thus formed was evaluated for entrapment efficiency which is 96.54%. Moreover, the particle size associated with this formulation is found to be in the range of 197 nm-207 nm. This formulation was stored at 4° C. until further use.

### Example 23

The nanostructured lipid carrier was prepared by hot homogenization followed by cold ultrasonication. The liquid phase consisting of weighed quantities of Monostearin (0.3% W/V), Labrafac Lipophile WL 1349 (0.15% W/V) and compound X (0.05% W/V) were heated to 90° ° C. with stirring. The aqueous phase consisting of Pluronic F68 (0.1% W/V) was heated to the same temperature and subsequently added to lipid phase and mixed using a homogenizer set at 13000 rpm for 5 min. The resulting coarse emulsion was ultrasonicated in an ice bath for 5 min. The suspension thus formed was evaluated for entrapment efficiency which is 94.47%. Moreover, the particle size associated with this formulation is found to be in the range of 2755 nm-4726 nm. This formulation was stored at 4° C. until further use.

### Example 24

The nanostructured lipid carrier was prepared by hot homogenization followed by cold ultrasonication. The liquid phase consisting of weighed quantities of Monostearin (0.3% W/V), Labrafac PG (0.15% W/V) and compound X (0.05% W/V) were heated to 90° C. with stirring. The aqueous phase consisting of Pluronic F68 (0.1% W/V) was heated to the same temperature and subsequently added to lipid phase and mixed using a homogenizer set at 13000 rpm for 5 min. The resulting coarse emulsion was ultrasonicated in an ice bath for 5 min. The suspension thus formed was evaluated for entrapment efficiency which is 88.38%. Moreover, the particle size associated with this formulation is found to be in the range of 504 nm-950 nm. This formulation was stored at 4° C. until further use.

### Example 25

The nanostructured lipid carrier was prepared by hot homogenization followed by cold ultrasonication. The liquid phase consisting of weighed quantities of Pricirol (0.3% W/V), Capryol90 (0.15% W/V) and compound X (0.05% W/V) were heated to 90° C. with stirring. The aqueous phase consisting of Pluronic F68 (0.1% W/V) was heated to the same temperature and subsequently added to lipid phase and mixed using a homogenizer set at 13000 rpm for 5 min. The resulting coarse emulsion was ultrasonicated in an ice bath for 5 min. The suspension thus formed was evaluated for entrapment efficiency which is 88.62%. Moreover, the particle size associated with this formulation is found to be in the range of 427 nm-471 nm. This formulation was stored at 4° C. until further use.

### Example 26

The nanostructured lipid carrier was prepared by hot homogenization followed by cold ultrasonication. The liquid phase consisting of weighed quantities of Pricirol (0.3% W/V), Labrafac Lipophile WL 1349 (0.1% W/V) and compound X (0.05% W/V) were heated to 90° ° C. with stirring. The aqueous phase consisting of Pluronic F68 (0.1% W/V) was heated to the same temperature and subsequently added to lipid phase and mixed using a homogenizer set at 13000 rpm for 4 min. The resulting coarse emulsion was ultrasonicated in an ice bath for 5 min. The suspension thus formed was evaluated for entrapment efficiency which is 97.02%. Moreover, the particle size associated with this formulation is found to be in the range of 995 nm-4285 nm. This formulation was stored at 4° C. until further use.

### Example 27

### Compound X Pharmacokinetic Study

Healthy male SD rats (weighing 200-230 gm) were used for the pharmacokinetic study. The animals were fasted overnight and had free access to water. The animals were divided into six groups with each group having 6 male SD rats (6×6=36) and were orally administered with optimized compound X nano lipid carrier formulation and coarse suspension (free drug) of compound X. All the formulations were administered orally with the help of rat oral feeding tube. The group comprises as follows, GROUP 1: Only vehicle sodium suspension as control/blank. GROUP 2: Compound X optimized NLC formulation by oral delivery (25 mg/kg). GROUP 3: Compound X free drug as coarse suspension by oral delivery (25 mg/kg). GROUP 4: Compound X free drug as coarse suspension by oral delivery (50 mg/kg). GROUP 5: Compound X free drug as coarse suspension by oral delivery (100 mg/kg). GROUP 6: Compound X free drug as coarse suspension by oral delivery (200 mg/kg). Blood samples were withdrawn from retro-orbital venous plexus puncture at time intervals of 0.25, 0.5, 1, 2, 4, 8, 12, 24 and 48 hours. The blood samples were allowed to clot and centrifuged for 15 min at 4500 rpm. The serum was separated and transferred into clean micro centrifuge tubes and stored at −80° C. until further analysis. 50 μl of rat serum was taken from samples and added to extraction solvent 2.5% IPA in n-Hexane (2 mL) containing internal standard (SCTK-14 of 2.5 ng) than Vortexed for 10 minutes, the samples were centrifuged at 4500 rpm and 4° C. for 15 minutes. After centrifugation, samples were stored at −80° C. for 30 minutes, and the supernatant organic layer was transferred and evaporated to dryness in a thermostatically controlled water bath maintained at 30° C. under steam of nitrogen for 30 minutes. After drying the residue was reconstituted by adding 200 microlitres of acetonitrile into pre-labelled vials and vortexed for 20 seconds, and then the samples were injected into the LC-MS system for analysis.

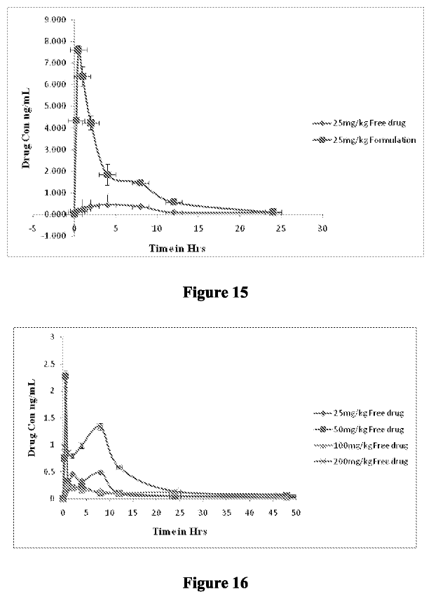

The pharmacokinetic parameters of compound X were calculated by non-compartmental estimations using PK solver 2.0 software. From above results, it was found that the absorption of NLC formulation is rapid and resulted in almost 10 fold enhancement in Cmax of compound X while AUC (area under curve) of compound X was enhanced by 5 fold when administered at the dose of 25 mg/kg body weight (Table 4, FIG. 15). However, when tested with free compound X at 25, 50, 100 and 200 mg/kg body weight there was no appreciable difference in AUC except at 200 mg/kg dose which was found to be ˜20 ng/ml*hr (Table 5, FIG. 16). It is clear that AUC of compound X NLC formulation at 25 mg/kg dose was significantly higher even when compared with 200 mg/kg free compound X dose.

TABLE 4
Pharmacokinetic Parameters of compound X NLC
formulation vs free drug

| | | 25 mg/kg NLC |
|---|---|---|
| Parameter | 25 mg/kg_Free Drug | Formulation |
| t½ (h) | 14.667 ± 5.07 | 4.101 ± 0.43 |
| Tmax (h) | 4.667 ± 3.05 | 0.667 ± 0.29 |
| Cmax (ng/ml) | 0.853 ± 0.09 | 7.830 ± 0.83 |
| Clast_obs/Cmax | 0.049 ± 0.04 | 0.013 ± 0.00 |
| AUC 0-t (ng/ml*h) | 6.561 ± 1.57 | 31.509 ± 3.17 |
| AUC 0-inf_obs (ng/ml*h) | 7.544 ± 0.80 | 32.112 ± 3.30 |

TABLE 5
Pharmacokinetic Parameters of Free LTK-14A at different doses

| | 25 mg/kg_ | 50 mg/kg_ | 100 mg/kg_ | 200 mg/kg_ |
|---|---|---|---|---|
| Parameter | Free Drug | Free Drug | Free Drug | Free Drug |
| t½ (h) | 14.667 ± 5.07 | 18.628 ± 12.24 | 13.781 ± 3.74 | 13.449 ± 4.31 |
| Tmax (h) | 4.667 ± 3.05 | 0.333 ± 0.14 | 0.667 ± 0.28 | 2.917 ± 4.40 |
| Cmax (ng/ml) | 0.853 ± 0.09 | 2.487 ± 0.14 | 0.343 ± 0.15 | 2.830 ± 2.16 |
| Clast_obs/Cmax | 0.049 ± 0.04 | 0.134 ± 0.14 | 0.045 ± 0.035 | 0.033 ± 0.02 |
| AUC 0-t (ng/ml*h) | 6.561 ± 1.57 | 4.744 ± 0.10 | 5.286 ± 0.99 | 19.145 ± 7.84 |
| AUC 0-inf_obs | 7.544 ± 0.80 | 6.493 ± 1.71 | 5.744 ± 1.60 | 20.563 ± 7.98 |
| (ng/ml*h) | | | | |
| MRT 0-inf_obs(h) | 22.246 ± 9.59 | 28.433 ± 22.77 | 20.963 ± 3.98 | 15.679 ± 2.87 |
| Vz/F_obs | 71.906 ± 29.05 | 193.686 ± 112.43 | 346.653 ± 2.6193 | 209.737 ± 91.22 |
| (mg)/(ng/ml) | | | | |
| Cl/F_obs | 3.338 ± 0.33 | 8.071 ± 2.12 | 18.233 ± 4.3 | 10.882 ± 4.58 |
| mg)/(ng/ml)/h | | | | |

1. A compound of formula (I) 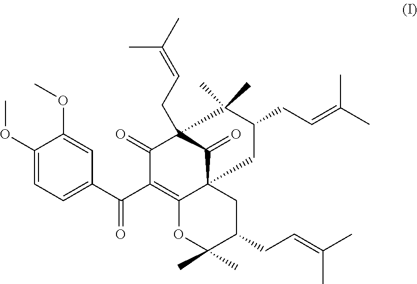
wherein the compound is useful as an anti-obesity agent by inhibiting p300 catalysed histone butyrylation without affecting acetylation and thereby specifically inhibiting adipogenesis without toxicity based side effects.

2. A process for preparation of the compound of Formula I as claimed in claim 1, comprising the steps of:
a. isolation of garcinol from Garcinia indica and synthesis of isogarcinol;
b. adding anhydrous potassium carbonate to a stirred solution of isogarcinol in dry acetone followed by dropwise addition of dimethyl sulfate under nitrogen gas atmosphere to obtain a reaction mass;
c. evaporating acetone and acidifying the reaction mass to obtain a precipitate;
d. filtering and washing the precipitate and subjecting to chromatography on silica gel using eluting solvent mixture; and
e. isolating the compound of Formula I as white solid.

3. The process as claimed in claim 2, wherein the eluting solvent mixture is 5-6% ethyl acetate in hexane.

4. A pharmaceutical composition for oral administration comprising,
i. the compound of Formula I as claimed in claim 1, 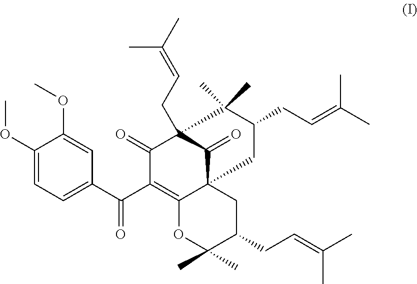
and
(ii) a pharmaceutically acceptable excipient,
wherein, the pharmaceutically acceptable excipient is selected from a group consisting of a lipid, a non-ionic surfactant, and a polymer; and the ratio of compound of Formula I to the excipient is 1:2.

5. The pharmaceutical composition as claimed in claim 4, wherein the amount of the lipid is 0.15 to 0.4% w/v, the amount of the non-ionic surfactant is 0.05 to 0.3% w/v, and the amount of the polymer is 0.05 to 0.25% w/v of the pharmaceutical composition.

6. The pharmaceutical composition as claimed in claim 4, wherein the lipid is selected from a group consisting of monoglycerides, diglycerides, triglycerides, phosphatidylcholine, di-stearoyl phosphatidylcholine, di-stearoyl phosphatidylglycerol, and cholesterol.

7. The pharmaceutical composition as claimed in claim 4, wherein the non-ionic surfactant is selected from a group consisting of poloxamers, cremophor, and polysorbates.

8. The pharmaceutical composition as claimed in claim 4, wherein the polymer is a pluronic polymer.

9. A compound of Formula (I), 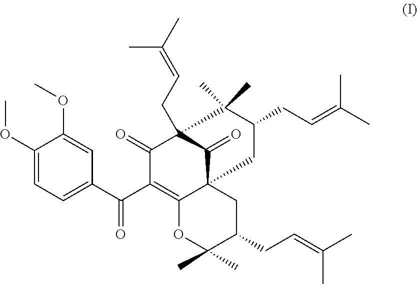
for use as a medicament for inhibiting adipogenesis.

10. A method of inhibiting adipogenesis by administering a therapeutically effective amount of a compound of Formula I, [chemical structure]
